(19) United States
(12) Patent Application Publication
(10) Pub. No.: US 2009/0124848 A1
(43) Pub. Date: May 14, 2009

(54) Receptacles for Implanted Device Control Magnets, and Associated Systems and Methods

(75) Inventor: Jay Miazga, Seattle, WA (US)

Correspondence Address: PERKINS COIE LLP;PATENT-SEA, P.O. BOX 1247, SEATTLE, WA, 98111-1247, US

(73) Assignee: Northstar Neuroscience, Inc., Seattle, WA (US)

(21) Appl. No.: 12/133,903

(22) Filed: Jun. 5, 2008

Related U.S. Application Data
(60) Provisional application No. 60/942,193, filed on Jun. 5, 2007.

(51) Int. Cl. A61N 2/00

(52) U.S. Cl. 600/12

(57) ABSTRACT

Receptacles for implanted device control magnets, and associated systems and methods are disclosed. A system in accordance with one embodiment includes a magnet receptacle that in turn has a first portion with a first magnetically conductive material and a second portion with a second magnetically conductive material. At least one of the first and second portions can be moveable relative to the other between a closed configuration and an open configuration, with the first and second portions forming a cavity when in the closed configuration, and with the first magnetically conductive material proximate to a first side of the cavity and the second magnetically conductive material proximate to a second side of the cavity facing toward the first side.

### CROSS-REFERENCE TO RELATED APPLICATION

The present application claims the benefit of U.S. Provisional Application 60/942,193, filed Jun. 5, 2007, and incorporated herein by reference.

### TECHNICAL FIELD

The present disclosure is directed generally toward receptacles for patient-implanted device control magnets, and associated systems and methods.

### BACKGROUND

Many patient treatment devices include control systems that are implanted in the patient. Representative treatment devices include cardiac stimulation devices and neural stimulation devices. Typically, the implanted control system is programmed by a practitioner or other medical professional prior to implantation, and then operates autonomously from within the patient after implantation. In some cases, the control system can be periodically updated using an external controller that communicates with the implanted control system via a magnetic or radio frequency (RF) link. Accordingly, the implanted control system can be updated without the need for a surgical procedure.

In many instances, it is desirable to allow the patient to override the implanted control system to perform specific functions on an as-needed basis. Such functions can include stopping the control system and/or starting the control system. To facilitate such non-automated operation, the patient can be provided with a special-purpose magnet that magnetically triggers a reed switch or other magnetically-sensitive device carried by the implanted control system. In one arrangement, the patient magnet is worn on a wristband, and in another arrangement, the magnet is carried in a soft pouch. While these arrangements have proven suitable for providing intermittent control over the implanted control system, they can suffer from several drawbacks. For example, the strength of the magnetic field created by the magnet may be such that it attracts ferromagnetic objects in an undesirable manner. If the patient wears the magnet on his or her wrist, the presence of the magnet can interfere with the patient's performance of manual tasks. If the magnet is placed in a purse or briefcase, the magnet can destroy credit card information, and/or other computer-based information. Still another drawback with existing devices is that they may require extra care and control for shipping, so as to reduce the potential impact on magnetically sensitive materials with which they may be shipped. Accordingly, there is a need for improved magnet systems for controlling patient-implanted devices.

### BRIEF DESCRIPTION OF THE DRAWINGS

FIG. 1 is a partially schematic, isometric illustration of a system that includes a magnet receptacle shown in its open position in accordance with an embodiment of the invention.

FIG. 2 is an isometric illustration of the receptacle shown in FIG. 1 in a partially closed position in accordance with an embodiment of the invention.

FIG. 3 is a partially schematic, isometric illustration of the magnet receptacle shown in FIGS. 1 and 2 in a closed position in accordance with an embodiment of the invention.

FIG. 4 is a partially schematic, exploded illustration of components used for manufacturing a magnet receptacle in accordance with an embodiment of the invention.

FIG. 5 is a partially schematic, side view of a magnet receptacle configured in accordance with another embodiment of the invention.

FIG. 6 is a schematic illustration of an implanted device configured in accordance with an embodiment of the invention.

FIG. 7 is a schematic illustration of internal features of an embodiment of the implanted device shown in FIG. 6.

FIG. 8 is a partially schematic, isometric illustration of an electrode device configured in accordance with an embodiment of the invention.

FIG. 9 is a partially schematic, side cross-sectional illustration of an electrode device configured in accordance with another embodiment of the invention.

### DETAILED DESCRIPTION

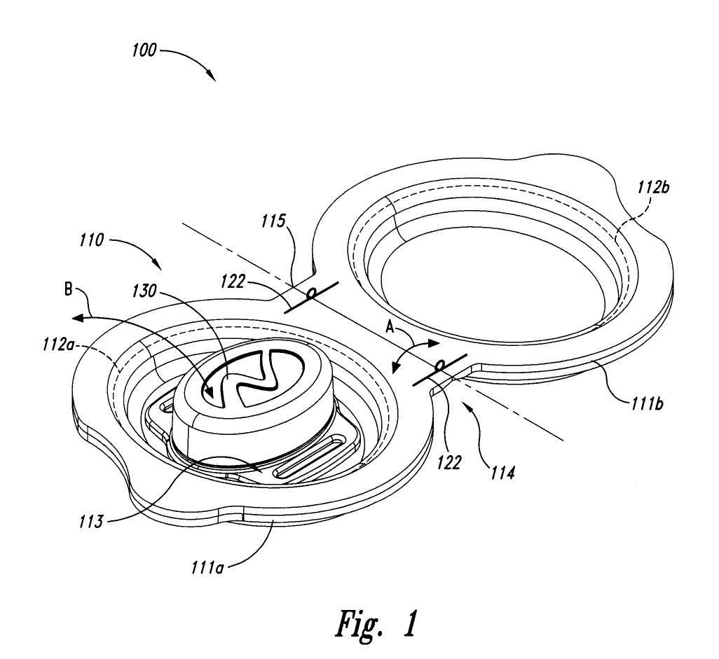

The present disclosure is directed generally toward receptacles for patient-implanted device control magnets, and associated systems and methods. Several of the details describing structures or processes that are well-known and often associated with aspects of these systems and methods are not set forth in the following description for purposes of brevity. Moreover, although the following disclosure sets forth several embodiments of different aspects of the invention, several other embodiments can have different configurations or different components than those described in this section. Such embodiments may have additional elements or may lack several of the elements described below with reference to FIGS. 1-9.

FIG. 1 is a partially schematic, isometric illustration of a magnet receptacle 110 that can form part of an overall patient treatment system 100 in accordance with an embodiment of the invention. In the illustrated embodiment, the magnet receptacle 110 includes two portions 111, shown as a first portion 111a and a second portion 111b. The two portions 111a, 111b can be connected by a coupling 114, e.g., a hinge 115. Accordingly, the two portions 111a, 111b can rotatably move toward and away from each other, as indicated by arrow A.

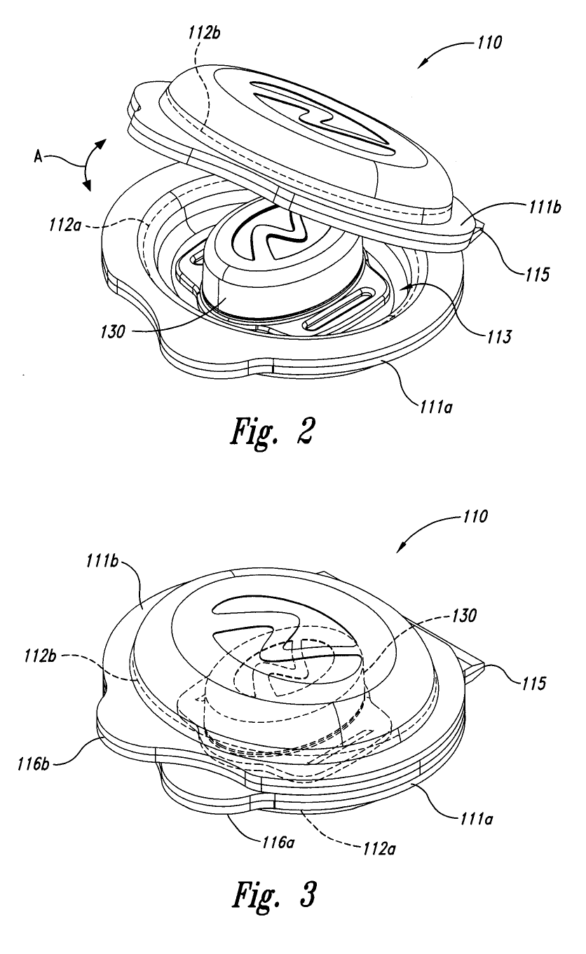

A cavity 113 is positioned between (and can be at least partially defined or formed by) the two portions 111a, 111b so as to removably house a magnet 130. Accordingly, when the two portions 111a, 111b are moved away from each other to an open configuration (shown in FIG. 1), the magnet 130 can be removed from the cavity 113 or placed back in the cavity 113, as indicated by arrow B. In the fully open configuration, the two portions 111a, 111b can face in the same direction. When the portions 111a, 111b are moved toward each other to a closed configuration (discussed further below with reference to FIGS. 2 and 3), the cavity 113 surrounds or at least partially surrounds the magnet 130.

The magnet receptacle 110 can further include one or more magnetically conductive materials that can provide shielding for the magnet 130 and/or secure the magnet 130 in the magnet receptacle 110. For example, in an embodiment shown in FIG. 1, the magnet receptacle 110 can include a first magnetically conductive material 112a (shown schematically in dashed lines) carried by the first portion 111a, and a second magnetically conductive material 112b (also shown schematically in dashed lines) carried by the second portion 111b. As used herein, magnetically conductive materials include materials that are magnetically responsive, e.g., magnetically permeable materials. The magnetically conductive materials 112a, 112b can have the same composition or different compositions, and can include any of a wide variety of ferromagnetic materials or other materials that are attracted to the magnet 130. Representative materials include steel, mu-metals, and/or other materials with high nickel content (e.g., about 75% nickel or greater), and/or other materials having high magnetic permeabilities (e.g., μ=0.01 N/A² or less). When the magnet receptacle 110 is in the open configuration shown in FIG. 1, the user can grasp the magnet 130 and pull with enough force to separate the magnet 130 from the first portion 111a. The user can then operate the magnet 130 to control a patient-implanted device. In another embodiment, the user can operate the magnet 130 without removing it from the receptacle 110. In either case, when the magnet receptacle 110 is in its closed configuration, the attractive forces between the magnet 130 and the first and second portions 111a, 111b can secure the receptacle 110 in its closed configuration, as described in further detail below.

FIG. 2 is an isometric illustration of the magnet receptacle 110 shown in a partially open/partially closed configuration. In this configuration, attractive forces between the magnet 130 and the first conductive material 112a secure the magnet 130 within the cavity 113. Attractive forces between the magnet 130 and the second conductive material 112b apply a force tending to draw the second portion 111b toward the first portion 111a. When closing the receptacle 110, this force tends to keep the two portions 111a, 111b together. When opening the receptacle 110, the user overcomes this force to pivot the two portions 111a, 111b away from each other about the hinge 115.

FIG. 3 illustrates the magnet receptacle 110 in its closed configuration. In this configuration, the first and second conductive materials 112a, 112b can face in opposite directions (e.g., toward each other) to form an at least partial enclosure around the magnet 130 inside. The first and second conductive materials 112a, 112b are also drawn to the magnet 130 so as to secure the receptacle 110 in its closed configuration. The first portion 111a can include a first finger tab 116a, and the second portion 111b can include a second finger tab 116b. Accordingly, the user can force the two portions 111a, 111b apart by pushing the corresponding finger tabs 116a, 116b apart, with a mechanical advantage provided by the distance between the tabs 116a, 116b and the hinge 115.

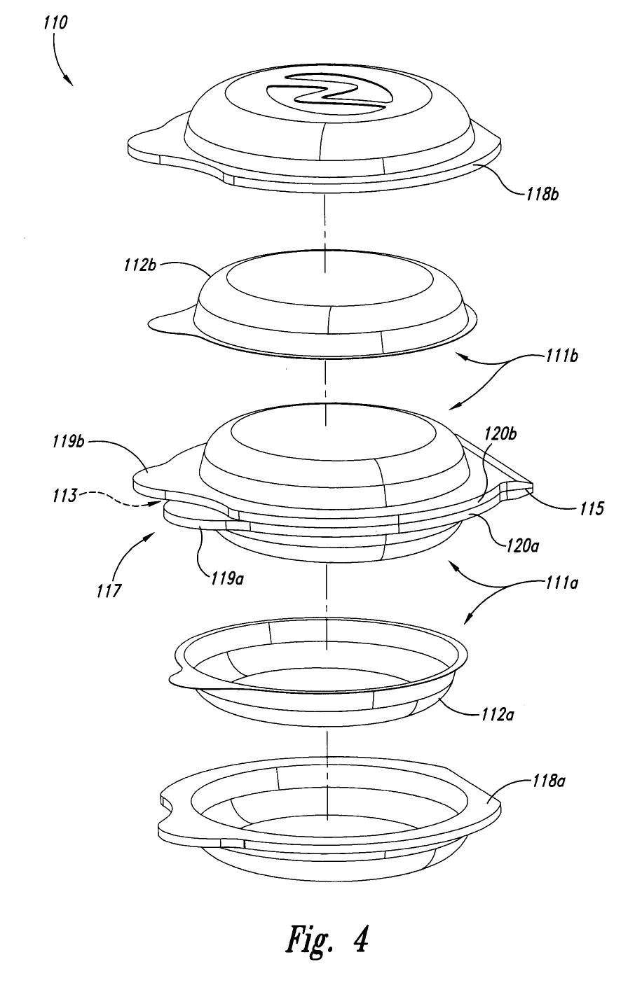

FIG. 4 is a partially schematic, exploded illustration of the receptacle 110, manufactured in accordance with a particular embodiment. In this embodiment, the receptacle 110 includes an inner member 117 that in turn includes a first section 119a and a second section 119b, coupled at the hinge 115. Each of the first and second sections 119a, 119b has a generally cup-shaped configuration that bounds the cavity 113. In a particular embodiment, the two sections 119a, 119b can be generally mirror images of each other. For example, the first and second sections 119a, 119b can be mirrored about a first (e.g., horizontal) plane that passes between the first and second sections 119a, 119b and through the hinge 115 to bisect the receptacle 113. The first and second sections 119a, 119b can also be mirrored about a second (e.g., vertical) plane that is perpendicular to the first plane and passes between the first and second finger tabs 116a, 116b so as to create an offset between the two finger tabs 116a, 116b.

The first section 119a can include a first flange 120a that projects outwardly from the cavity 113, and the second section 119b can include a corresponding second flange 120b. In a particular embodiment, the thicknesses of the flanges 120a, 120b can be reduced at the hinge 115 so that the first and second sections 119a, 119b and the hinge 115 can be formed from a unitary piece of material. In other embodiments, the hinge 115 can be a separate element that is attached to separate first and second sections 119a, 119b.

The first conductive material 112a can be positioned around the first section 119a, and can have a shape that generally conforms to the exterior surface of the first section 119a. Similarly, the second conductive material 112b can conform to the outer surface of the second section 119b. In this manner, the first conductive material 112a and the second conductive material 112b almost completely surround the cavity 113, except for a small circumferential band corresponding to the combined thicknesses of the first flange 120a and the second flange 120b. In other embodiments, the first and second conductive materials 112a, 112b can have other configurations that completely or nearly completely surround the cavity 113, thereby providing a significant shielding effect around the magnet 130 (FIG. 1) when it is positioned in the cavity 113.

The receptacle 110 can further include a first outer member 118a that is positioned around the first conductive material 112a, and a second outer member 118b that is positioned around the second conductive material 112b. The first and second outer members 118a, 118b, along with the inner member 117, can provide a resilient, compliant coating around the conductive materials 112a, 112b to protect the conductive materials from environmental factors (e.g., moisture) and protect the user from direct contact with the conductive materials 112a, 112b.

The dimensions of the inner member 117 and the outer members 118a, 118b can be selected to have a desired effect on a number of system parameters, including but not limited to, the level of shielding provided by the receptacle 110, the ease with which the receptacle 110 can be opened, and/or the force with which the receptacle 110 closes. For example, in a particular embodiment, the material thicknesses of the inner member 117 and the outer members 118a, 118b can be selected to be from about 0.080 inches to about 0.200 inches. The thicknesses can have other values in other embodiments, and in any of these embodiments, the selected values(s) can determine, at least in part, the operating characteristics of the receptacle 110. For example, selecting a low thickness value for the inner member 117, particularly at the flanges 120a, 120b can improve shielding by reducing the gap between the conductive materials 112a, 112b. This arrangement can also increase the closing force of the receptacle 110 because the spacing between the conductive materials 112a, 112b and the magnet 130 is reduced. Conversely, increasing the thickness value at the flanges 120a, 120b can increase the separation between the conductive materials 120a, 120b, which can reduce shielding, but can also reduce the force required to open the receptacle 110. In addition, increasing the thickness value for the inner member 117 (e.g., at least in regions away from the flanges 120a, 120b) can increase the separation between the magnet 130 and the conductive materials 112a, 112b. This in turn can improve the shielding effectiveness of the receptacle 110. In particular embodiments, the inner member 117 and the outer members 118a, 118b are each uniformly thick, and in other embodiments, these members can have different thicknesses, e.g., different thicknesses at the flanges 120a, 120b than at the inner regions of the cavity 113, depending on the desired shielding level, closing force and opening force. The inner member 117 can have the same thickness as the outer members 118a, 118b, or a different thickness, and each of the outer members 118a, 118b can have the same thickness or different thicknesses depending upon the particular design.

One feature of at least some of the foregoing embodiments is that they can include a magnet receptacle that includes magnetically conductive materials. The materials can be positioned so that they secure the magnet within the receptacle, secure the receptacle in a closed configuration, and/or provide a shield around the magnet once it is in the receptacle. This arrangement can have several advantages. For example, this arrangement can reduce the likelihood that the magnet within the receptacle will interfere with, damage, and/or otherwise adversely affect magnetically sensitive materials in its immediate environment. For example, the magnet by itself, without being enclosed by the receptacle, can have a field strength in the range of about 5-11 mTesla (measured 25 mm from the pole surfaces). When the magnet is within the receptacle, the field strength outside the receptacle can have a significantly lower value. Representative values include about 1 mTesla or less (e.g., 0.1 mTesla or less) measured just outside the receptacle, and/or about 0.0002 mTesla or less, measured 2.1 meters outside a shipping carton in which the magnet and receptacle are placed. In a representative embodiment, the magnet itself can have a field strength of about 10 mTesla as measured by a testing probe positioned in a selected orientation relative to the magnet, and spaced about 25 mm from the magnet. When the magnet is placed inside the receptacle, and the probe has the same orientation relative to the magnet and the same spacing from the magnet, the field strength can have a value of about 3.7 mTesla. Lower values outside the receptacle can be obtained by appropriately selecting the composition of the magnetically conductive materials 112a, 112b, the thickness of the sections 119a, 119b, and/or the orientation of the magnet 130 within the cavity 113. In any of these embodiments, the user can place the receptacle (with the magnet inside) in close proximity to credit cards, computer-readable media, ferromagnetic items, and other magnetically-sensitive materials, with the potential for interfering with the functionality of such materials or the receptacle reduced or eliminated. This arrangement can also have advantages for shipping, because the shielding can reduce or eliminate the need for carefully positioning the magnet away from materials that might be adversely affected by its presence.

Another advantage of the foregoing feature is that the receptacle need not include a separate closure, locking, and/or securement device (e.g., a mechanical latch). Instead, the magnetically conductive materials that provide the shielding described above can, in concert with the magnet, operate as a closure device. This arrangement can simplify the construction of the receptacle and can also provide advantages for its use. For example, in at least one embodiment, the receptacle cannot be fully secured unless the magnet is inside. As a result, if the user attempts to close the receptacle without the magnet inside, the user will be unable to secure the receptacle, providing a clear indication that the magnet is missing. The receptacle may also be “floppy” in that the first and second portions 111a, 111b easily move away from each other, providing both a visual and tactile indication that the receptacle is empty.

In a particular embodiment, the receptacle can include one or more forcing devices (e.g., springs 122 shown in FIG. 1) between the two portions 111a, 111b that tend to force the portions apart from each other. In this way, the receptacle will tend to be forced open unless the magnet is inside, thereby providing a clear visual indication that the magnet is missing. Another advantage of the spring is that it can make the receptacle easier to open because it applies a separating force between the two portions 111a, 111b.

Still another feature of at least some of the foregoing embodiments is that the materials forming the receptacle can be resilient and compliant. For example, they can include one or more plastic materials (e.g., a foam material) having any of a variety of degrees of softness and/or compliance. Generally, the materials can be softer than the magnet 130 and/or the magnetically conductive materials 112a, 112bb. For example, the material(s) forming the sections 119a, 119b, and/or the outer members 118a, 118b can include a polyester-covered thermoform foam (e.g., a thermoformed EVA (Ethylene-Vinyl Acetate) with a 600 denier polyester skin, available from Polo Custom Products of Topeka, Kans.). An advantage of this arrangement is that the resilient nature of the materials can prevent the user from coming into direct contact with the conductive materials, which may have sharp edges. Another advantage is that it can reduce the effect of the patient inadvertently closing the receptacle on his or her finger.

FIG. 5 is a partially schematic, top view of an enclosure 510 configured in accordance with another embodiment. In one aspect of this embodiment, the receptacle 510 includes a first portion 511a and a second portion 511b, each of which is pivotable relative to the other about a pivot axis 515 that extends generally out of the plane of FIG. 5. Accordingly, each of the first and second portions 511a, 511b can be pivoted relative to the other, as indicated by arrow C. When the receptacle 510 is closed, the portions 511a, 511b face toward each other in opposite directions and are laterally aligned with each other. When the receptacle 510 is open, the portions 511a, 511b still face in opposite directions, but are at least partially offset (laterally) from each other. When closed, the receptacle 510 includes a cavity 513 that houses and at least partially encloses, surrounds and/or shields the magnet 130. The cavity 513 can be positioned entirely in the first portion 511a so that the second portion 511b can slide over the magnet 130 without contacting it. Each of the portions 511a, 511b can include corresponding conductive materials and/or compliant, resilient materials, generally similar to those described above with reference to the FIGS. 1-4.

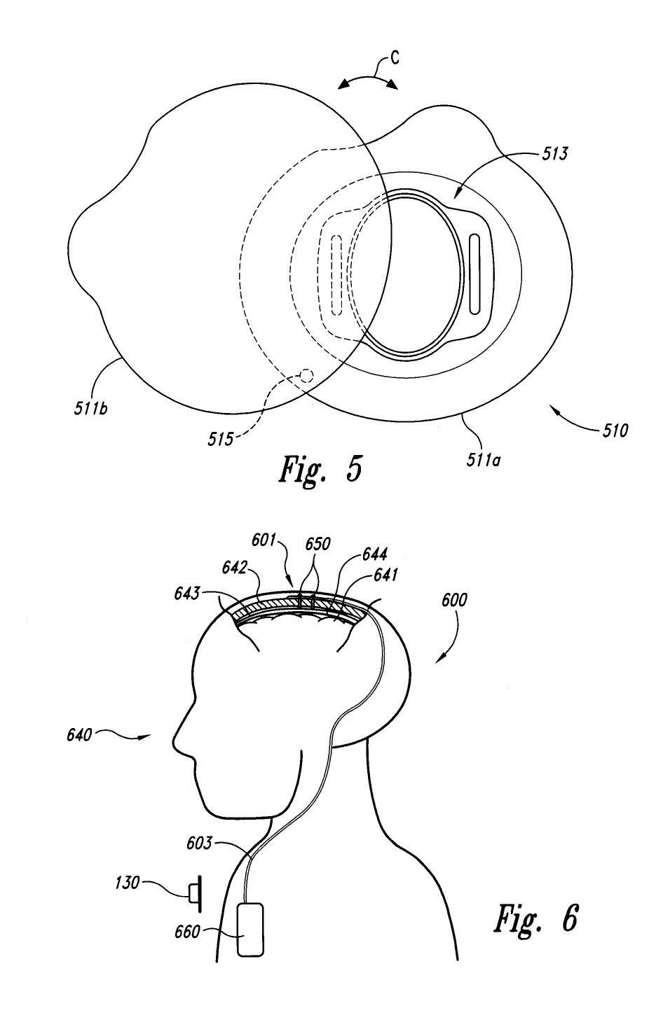

FIGS. 6-9 illustrate other aspects of an overall system that can include magnetic receptacles in accordance with any of the foregoing embodiments. In a particular embodiment, the overall system includes a neurostimulator having an electrode or other device that provides electromagnetic signals to a patient's brain. In other embodiments, the foregoing receptacles can be used to house magnets for controlling other types of neural devices, cardiac devices, or other devices.

FIG. 6 is a schematic illustration of a neurostimulation system 600 implanted in a patient 640 to provide electromagnetic signals (e.g., electromagnetic stimulation) in accordance with several embodiments. The system 600 can include an electrode device 601 carrying one or more electrodes 650. The electrode device 601 can be positioned in the skull 642 of the patient 640, with the electrodes 650 positioned to apply signals to target areas of the brain 641. For example, the electrodes 650 can be positioned just outside the dura mater 643 (which surrounds the brain 641) to direct signals to the cortical tissue. In another embodiment described later with reference to FIG. 9, an electrode can penetrate the dura mater 643 to apply signals to subcortical tissues. In still further embodiments, the electrodes 650 can penetrate the dura mater 643 but not the underlying pia mater 644, and can accordingly provide signals through the pia mater 644.

The electrode device 601 can be coupled to a pulse system 660 with a communication link 603. The communication link 603 can include one or more leads, depending (for example) upon the number of electrodes 650 carried by the electrode device 601. The pulse system 660 can direct electrical signals to the electrode device 601 to stimulate target neural tissues.

The pulse system 660 can be implanted at a subclavicular location, as shown in FIG. 6. The pulse system 660 can be controlled internally via pre-programmed instructions that allow the pulse system 660 to operate autonomously after implantation. In other embodiments, the pulse system 660 can be implanted at other locations, and at least some aspects of the pulse system 660 can be controlled externally. For example, the pulse system 660 can be controlled by the magnet 130, either when the magnet 130 is removed from the receptacle 110 (FIG. 1), or when the magnet 130 is in the receptacle 110 with the receptacle 110 in its open configuration. Further details regarding the external control of the pulse system 660 are described below.

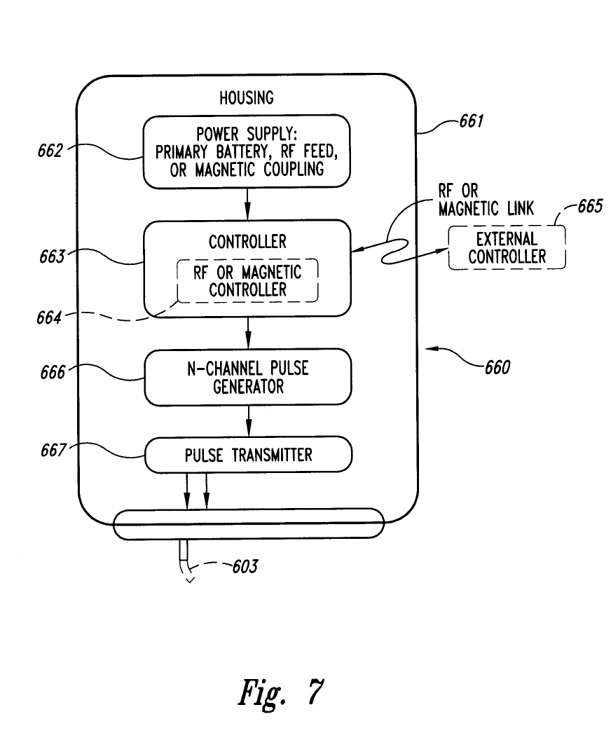

FIG. 7 schematically illustrates a representative example of the pulse system 660. The pulse system 660 generally includes a housing 661 carrying a power supply 662, an integrated controller 663, a pulse generator 666, and a pulse transmitter 667. The power supply 662 can be a primary battery, such as a rechargeable battery or other suitable device for storing electrical energy. In other embodiments, the power supply 662 can be an RF transducer or a magnetic transducer that receives broadcast energy emitted from an external power source and that converts the broadcast energy into power for the electrical components of the pulse system 660.

In one embodiment, the integrated controller 663 can include a processor, a memory, and a programmable computer medium. The integrated controller 663, for example, can be a microcomputer, and the programmable computer medium can include software loaded into the memory of the computer, and/or hardware that performs the requisite control functions. In another embodiment identified by dashed lines in FIG. 6, the integrated controller 663 can include an integrated RF or magnetic controller 664 that communicates with than external controller 665 via an RF or magnetic link. In such an embodiment, many of the functions performed by the integrated controller 663 may be resident on the external controller 665, and the integrated portion 664 of the integrated controller 663 may include a wireless communication system. The magnet 130 (described above with reference to FIGS. 1-5) provides one example of an external controller 665, that, in a particular embodiment, performs a limited number of functions (e.g., turning the power supply 662 and/or integrated controller 663 on and/or off).

The integrated controller 663 is operatively coupled to, and provides control signals to, the pulse generator 666, which may include a plurality of channels that send appropriate electrical pulses to the pulse transmitter 667. The pulse generator 666 may have multiple channels, with at least one channel associated with a particular one of the electrodes 650 (FIG. 6). The pulse generator 666 sends appropriate electrical pulses to the pulse transmitter 667, which is coupled to the electrodes 650. In one embodiment, each of the electrodes 650 is configured to be physically connected to a separate lead, allowing each electrode 650 to communicate with the pulse generator 666 via a dedicated channel. Suitable components for the power supply 662, the integrated controller 663, the external controller 665, the pulse generator 666, and the pulse transmitter 667 are known to persons skilled in the art of implantable medical devices.

The pulse system 660 can be programmed and operated to adjust a wide variety of signal delivery parameters, e.g., which electrodes are active and inactive, whether electrical signals are provided in a unipolar or bipolar manner, and/or how the signals are varied. In particular embodiments, the pulse system 660 can be used to control the polarity, frequency, duty cycle, amplitude, and/or spatial and/or temporal qualities of the signals. The signals can be varied to match naturally occurring burst patterns (e.g., theta burst stimulation), and/or the signals can be varied in a predetermined, pseudorandom, and/or aperiodic manner at one or more times and/or locations.

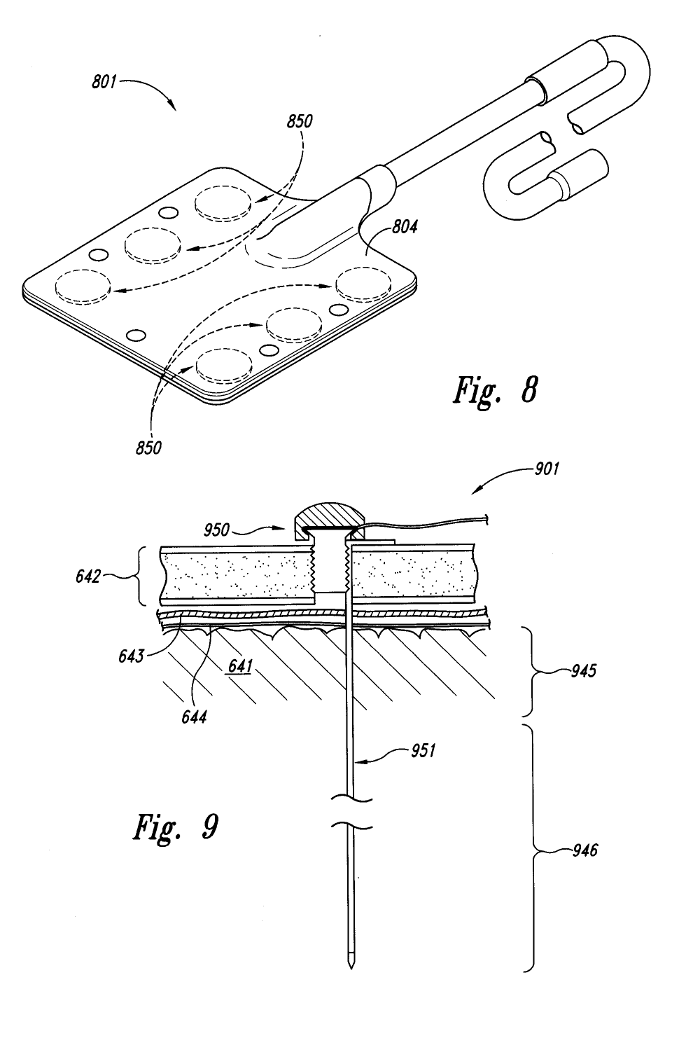

Electromagnetic signals can be provided to the patient using devices in addition to or in lieu of those described above. For example, FIG. 8 is a top, partially hidden isometric view of an electrode device 801 configured to carry multiple cortical electrodes 850 in accordance with a particular embodiment. The electrodes 850 can be carried by a flexible support member 804 (located within the patient's skull) to place each electrode 850 at a signal delivery site of the patient when the support member 804 is implanted within the patient's skull. In an embodiment shown in FIG. 8, the electrode device 801 can include six electrodes 850 arranged in a 2×3 electrode array (i.e., two rows of three electrodes each), and in other embodiments, the electrode device 801 can include more or fewer electrodes 850 arranged in symmetrical or asymmetrical arrays.

FIG. 9 illustrates an electrode device 901 that may be configured to apply electrical signals to a cortical region 945 or a subcortical region 946 of the brain 641 in accordance with further embodiments of the invention. If the electrode 950 is intended for cortical signal delivery, it can extend through the skull 642 to contact the dura mater 643 or the pia mater 644. If the electrode 950 is to be used for subcortical signal delivery, it can include an elongate conductive member 951 that extends downwardly through the cortical region 945 into the subcortical region 946.

From the foregoing, it will be appreciated that specific embodiments of the invention have been described herein for purposes of illustration, but that various modifications may be made without deviating from the invention. For example, the receptacle can have configurations and arrangements other than those specifically shown and described above. The configuration of the receptacle can depend at least in part on the configuration of the magnet. Accordingly, in other embodiments, the magnet can have a configuration other than that shown in the Figures, and the receptacle can have a corresponding configuration to suitably house the magnet. The receptacle (e.g., the inner and outer portions of the receptacle) can include materials other than those described above. In still further embodiments, the portions of the receptacle can rotate relative to each in manners other than those described above, and/or can move in manners that include non-rotational motion.

Certain aspects of the invention described in the context of particular embodiments may be combined or eliminated in other embodiments. Further, while advantages associated with certain embodiments of the invention have been described in the context of those embodiments, other embodiments may also exhibit such advantages, and not all embodiments need necessarily exhibit such advantages to fall within the scope of the invention.

I/We claim:

1. A system for controlling a patient therapy device, comprising:
a magnet receptacle that includes:
a first portion having a first magnetically conductive material; and
a second portion having a second magnetically conductive material, at least one of the first and second portions being movable relative to the other between a closed configuration and an open configuration, with the first and second portions forming a cavity when in the closed position, and with the first magnetically conductive material proximate to a first side of the cavity and the second magnetically conductive material proximate to a second side of the cavity facing toward the first side.

2. The system of claim 1, further comprising a magnet removably positioned in the cavity, the magnet being positioned to draw the first and second portions of the receptacle toward each other.

3. The system of claim 1, further comprising a forcing device coupled to the first and second portions and positioned to force the first and second portions toward the open configuration.

4. The system of claim 1 wherein the magnet receptacle does not include a latch positioned to secure the first and second portions in the closed position.

5. The system of claim 1 wherein each of the first and second portions includes a non-conductive protective material disposed adjacent to the corresponding magnetically conductive material, and wherein the magnetically conductive material includes a layer of ferromagnetic material.

6. The system of claim 5 wherein the protective material is compliant.

7. The system of claim 5 wherein the protective material faces inwardly toward the cavity when the magnet receptacle is in the closed position.

8. The system of claim 5 wherein the protective material faces outwardly away from the cavity when the magnet receptacle is in the closed position.

9. The system of claim 1 wherein the first and second portions are rotatably connected to each other.

10. The system of claim 9 wherein the first and second portions face the same direction when in the open configuration, and wherein the first and second portions face toward each other in opposite directions when in the closed configuration.

11. The system of claim 9 wherein the first and second portions are at least partially offset from each other in lateral direction and face in opposite directions when in the open configuration, and wherein the first and second portions face toward each other in opposite directions when in the closed configuration.

12. The system of claim 1 wherein first and second portions are unitary and wherein a hinge between the first and second portions includes a portion of material having a thickness less than a thickness of the first and second portions.

13. The system of claim 1 wherein the first portion includes a first finger tab projecting outwardly from the cavity when the first and second portions are in the closed configuration, and wherein the second portion includes a second finger tab at least partially offset from the first finger tab and projecting outwardly from the cavity when the first and second portions are in the closed configuration.

14. The system of claim 1 wherein the first and second potions are mirrored relative to each other about a first plane that passes between the first and second portions and bisects the cavity when the first and second portions are in the closed configuration.

15. The system of claim 14 wherein the first and second portions are mirrored relative to each other about a second plane that is perpendicular to the first plane.

16. A system for controlling a patient therapy device, comprising:
a magnet receptacle that includes:
a first portion enclosing a first magnetically conductive material and having a first finger tab;
a second portion enclosing a second magnetically conductive material and having a second finger tab laterally offset from the first finger tab, the first and second portions being rotatably coupled to each other and movable relative each other between a closed configuration and an open configuration, with the first and second portions forming a cavity when in the closed position, the cavity being within a region bounded at least in part by the first magnetically conductive material on a first side and by the second magnetically conductive material on a second side facing toward the first side; and
a magnet removably positioned in the cavity, the magnet being positioned to draw the first and second portions of the receptacle toward each other.

17. The system of claim 16 wherein the first and second portions face the same direction when in the open configuration, and wherein the first and second portions face toward each other in opposite directions when in the closed configuration.

18. The system of claim 16 wherein the first and second portions are at least partially offset from each other in a lateral direction and face in opposite directions when in the open configuration, and wherein the first and second portions face toward each other in opposite directions when in the closed configuration.

19. The system of claim 16 wherein a field strength resulting from the magnet outside the receptacle is in the range of from about 5-11 mTesla, measured 25 mm from the magnet, and wherein a field strength resulting from the magnet within the receptacle when the first and second portions are in the closed configuration is about 0.1 mTesla or less, measured just outside the receptacle.

20. The system of claim 16 wherein:
the first and second portions face the same direction when in the open configuration, and wherein the first and second portions face toward each other in opposite directions when in the closed configuration;
the first and second potions are mirrored relative to each other about a first plane that passes between the first and second portions and bisects the cavity when the first and second portions are in the closed configuration;
the first and second portions are mirrored relative to each other about a second plane that is perpendicular to the first plane;
each of the first and second magnetically conductive materials includes a layer of ferromagnetic material;
each of the first and second portions includes a non-conductive protective material disposed adjacent to inwardly and outwardly facing surfaces of the corresponding magnetically conductive material, the protective material being compliant and softer than both the magnetically conductive material and the magnet;
the magnet receptacle does not include a latch positioned to secure the first and second portions in the closed position; and wherein
the system further comprises a spring coupled to the first and second portions and positioned to force the first and second portions away from each other toward the open configuration.

21. A method for using a patient treatment device, comprising:
placing a magnet in an open magnet receptacle;
closing, securing, or both closing and securing the magnet receptacle by applying a magnetic force on the receptacle with the magnet;
opening the magnet receptacle;
removing the magnet from the magnet receptacle; and
using the magnet to control an implanted patient device.

22. The method of claim 21 wherein the magnet receptacle includes a first portion and a second portion that is rotatable relative to the first portion, and wherein the method further comprises:
securing the two portions of the receptacle in a closed position with the magnet by applying a magnetic force on ferromagnetic components of the two portions; and
shielding a region external to the receptacle from the magnetic force via the ferromagnetic components of the two portions.

23. The method of claim 21 wherein closing, securing, or both closing and securing the magnet receptacle includes securing the receptacle in a closed configuration without a latch.

24. The method of claim 21 wherein the magnet receptacle includes a first portion and a second portion that is rotatable relative to the first portion, and wherein opening the receptacle includes rotating at least one of first and second portions relative to the other.

25. The method of claim 24 wherein rotating includes rotating the at least one portion relative to the other from a closed configuration in which the first and second portions face toward each other in opposite directions, to an open configuration in which the two portions face in the same direction.

26. The method of claim 24 wherein rotating includes rotating the at least one portion relative to the other from a closed configuration in which the first and second portions face toward each other in opposite directions, to an open configuration in which the two portions face in opposite directions and are at least partially offset from each other in a lateral direction.

27. The method of claim 21 wherein using the magnet includes using the magnet to turn the implanted patient device on.

28. The method of claim 21 wherein using the magnet includes using the magnet to turn the implanted patient device off.

29. The method of claim 21 wherein using the magnet includes using the magnet to control a sub-clavicularly positioned implanted pulse generator coupled to a cortical electrode positioned beneath a patient's skull.
